SEQUENCE LISTING (4 sequences)

SEQ ID NO: 1 - DNA, 1058 bp, Anser sp.
  source 1..1058: mol_type = genomic DNA
catgtgcagccaaaactaatgcataaaatcatacacttgcctgcaagcattacatggatagccctggcttagctgctgaagaagcaggctgctgcttttagcagcagttatagcaagagcttcaagacaaataacattcccttgagggacagggccatacaggttcagaactgaaggtattctggaaaatgcactttaagcatcatgcacaatgcagtacctaactggagtgtactaagagctaagacttaccagtctggagacaacagacttgattccagaaggaagtgataatttatcttgctctcttagtattacacttgacctagctttaatgagcaagatctagatatgtcgtgacgcatccttttgggtatactaaggaatatctctgccctcatttgtggggacaaccagatggtagcaactaactttgtccattattataactttccaggcttcaggactactactgctgaagtaacttatatcaggaaaaaatggtgtatgtttaaaaaaaaatagaacaggtatctaagctatctttagagacatcttaggatggtaatgtatgagcaggggagtactgaattcagctcagcattagtcaagtttaaactgtttgctaaaagtctgaggatcttttcgaatgttatgttaggttgtcgacagcaggagcctgtgcacaagcatccttttcccttgcaaactagaccctaccttaaaaaggaaagctcagtaataggaacactgagggtggtttactactatctgacgagtatactgtcatactgcttaatctctgcacttgtactatgatggaaagcttctggttctgctgtcatagttgaataaggtaactacaatcaaaagattcagagatcagatatagtttcatgctgatgttatgctgtccattaagctggttatctacttgatcagcagtgggtgaaagtgaaggaaaatactgctgaacagtactggctcagattctttttttcattgaaggtgcgagagcaaaatgtgaagaatcccagttcccgtgtagtaatggacgc

SEQ ID NO: 2 - DNA, 445 bp, Anser sp.
  source 1..445: mol_type = genomic DNA
gggacagggccatacaggttcagaactgaaggtattctggaaaatgcactttaagcatcatgcacaatgcagtacctaactggagtgtactaagagctaagacttaccagtctggagacaacagacttgattccagaaggaagtgataatttatcttgctctcttagtattacacttgacctagctttaatgagcaagatctagatatgtcgtgacgcatccttttgggtatactaaggaatatctctgccctcatttgtggggacaaccagatggtagcaactaactttgtccattattataactttccaggcttcaggactactactgctgaagtaacttatatcaggaaaaaatggtgtatgtttaaaaaaaaatagaacaggtatctaagctatctttagagacatcttaggatggtaatgtatgagcaggggagtactga

SEQ ID NO: 3 - DNA, 20 bp, synthetic construct
  misc_feature 1..20: note = Artificially designed primer sequence
  source 1..20: mol_type = other DNA
gggacagggccatacagttt

SEQ ID NO: 4 - DNA, 22 bp, synthetic construct
  misc_feature 1..22: note = Artificially designed primer sequence
  source 1..22: mol_type = other DNA
tcagtactcccctgctcataca

(19) United States
(12) Patent Application Publication
(10) Pub. No.: US 2023/0193404 A1
(43) Pub. Date: Jun. 22, 2023

(54) Molecular Marker for Identifying Poultry Laying Traits Based on OVR Gene and Identification Method and Application Thereof

(71) Applicant: Anhui Agricultural University, Hefei (CN)

(72) Inventors: Xingyong CHEN, Hefei (CN); Yeye DU, Hefei (CN); Zhengquan LIU, Hefei (CN)

(21) Appl. No.: 18/049,750

(22) Filed: Oct. 26, 2022

(30) Foreign Application Priority Data
Oct. 26, 2021 (CN) 202111246652.X

(51) Int. Cl. C12Q 1/6888

(52) U.S. Cl. CPC C12Q 1/6888; C12Q 2600/124; C12Q 2600/156

(57) ABSTRACT

The present disclosure discloses a molecular marker for identifying poultry laying traits based on an OVR gene, an identification method and an application thereof. The molecular marker is a polymorphic site which is developed on the basis of an oocyte vitellogenesis receptor (OVR) gene and capable of affecting the expression quantity thereof. The expression of the OVR gene is affected by the binding efficiency of C/EBPα to the transcriptional control region. The molecular marker is a G/T mutation located at −399 bp upstream of the transcription initiation site of the OVR gene; the type of the molecular marker is identified in the poultry genome to judge the egg laying capacity of an individual poultry via molecular screening at the early stage, which provides direct technical means for the breeding of poultry egg laying capacity.

### CROSS-REFERENCE TO RELATED APPLICATION

This application claims priority from the Chinese patent application 202111246652.X filed Oct. 26, 2021, the content of which is incorporated herein in the entirety by reference.

### INCORPORATION-BY-REFERENCE OF SEQUENCE LISTING OR TABLE

The material in the accompanying sequence listing is hereby incorporated by reference in its entirety into this application. The accompanying file, named “10_143AY_010USU_Sequence_Listing.xml” was created on Oct. 26, 2022, and is 6.73 KB.

### TECHNICAL FIELD

The present disclosure relates to the technical field of molecular markers, and in particular to a molecular marker for identifying poultry laying traits based on an OVR gene and an identification method and an application thereof.

### BACKGROUND

As one of reproductive traits, egg production is affected by follicular maturation and ovulation; the follicular development and maturation are crucial to the synthesis and transporting adjustment of a yolk precursor. Yolk precursor mainly includes very low density lipoprotein (VLDLy) and vitellogenin (VTG). Female poultry secretes estrogen to stimulate liver to synthesize yolk precursor, and thus, yolk precursor is released into blood circulation, and transferred to ovary for follicle formation. During the rapid development stage of follicle, VLDLy, VTG and nutrient substances enter into the granule cell layer of theca folliculi via blood vessels on the surface of theca folliculi, and then are bound with oocyte vitellogenesis receptor (OVR), located on the surface of granule cells. The OVR bound yolk precursors are subjected to endocytosis into follicle membrane to support the development of oocytes. Subsequently, the ligand is isolated from the receptor complex, and the receptor gets back to the surface of oocyte for the next round of transportation. The isolated VLDLy is degraded under the action of a lysozyme, and thus absorbed by the oocyte to form yolk substances. Thus, it can be seen that yolk development mainly depends on the quantity of VLDLy entering into the oocyte; that is, the expression quantity of the receptor OVR/VLDLR on the surface of the granule cell layer determines the content of yolk substances and the development rate of follicle.

In the early stage of egg laying, the synthesis of yolk substances is regulated by hormones, while the expression of the OVR on the surface of theca folliculi granule cells is mainly regulated by the C/EBPα binding in the transcriptional control region instead of hormones. Therefore, the polymorphism of the gene sequence in the control region of the OVR gene expression directly affects the expression quantity, thus affecting the development of follicle.

As one of the reproductive traits, the egg production has a lower hereditary capacity in the male parent family Moreover, as an economic character, the egg production is controlled by micro-effect polygenes. The egg production of an individual poultry has been always used as the major selection basis, leading to slow breeding progress. Point mutation of the OVR gene can lead to the limited egg production of leghorn poultry. As can be seen, the OVR gene directly affects the egg production via promoting the follicular development. Therefore, the OVR gene is chosen as a candidate gene and the expression transcription factor binding site thereof serves as a study region to screen the molecular marker capable of being used for the breeding of egg production character, which is a direct technical means for poultry breeding. Based on the above contents, the present disclosure provides a molecular marker for identifying poultry laying traits based on an OVR gene and an identification method and an application thereof.

### SUMMARY

Directed to the poultry reproductive trait of low hereditary capacity, the objective of the present disclosure is to provide a molecular marker for identifying poultry laying traits based on OVR gene, thus achieving early detection by means of the molecular marker technology, and overcoming the technical problem of time-consuming phenotype recording.

The objective of the present disclosure is achieved by the following technical solutions:

The present disclosure provides a molecular marker for identifying poultry laying traits based on an OVR gene; the molecular marker is G or T, and the molecular marker is located at -399bp upstream of a transcription initiation site of an oocyte vitellogenesis receptor (OVR) gene. The molecular marker is a polymorphic site which is developed on the basis of an oocyte vitellogenesis receptor (OVR) gene and capable of affecting the expression quantity thereof. The expression of the OVR gene is affected by the binding efficiency of C/EBPα to the transcriptional control region. The molecular marker is located in the C/EBPα transcriptional control binding region, and is specifically a G/T mutation located at -399bp upstream of the transcription initiation site of the OVR gene.

The sequence as shown in SEQ ID NO.1 is partial OVR gene sequences and upstream sequence thereof; the transcription initiation site (ATG) of the OVR gene is located at sites 816-818 of the sequence as shown in SEQ ID NO.1; the molecular marker is located at −399bp upstream of the transcription initiation site of the OVR gene, namely, located at a 417th site of the nucleotide sequence as shown in SEQ ID NO.1.

The present disclosure further provides an application of the above molecular marker for identifying poultry laying traits of based on an OVR gene in identifying poultry laying traits.

The present disclosure further provides a method for identifying poultry laying traits by using the above molecular marker, including the following steps of:

(1) extracting poultry blood and total DNA of any one tissue;

(2) serving OVR nucleotide sequences upstream and downstream of the site where the molecular marker is located as templates to design specific amplimers; serving the total DNA as a template and using the specific amplimers for PCR amplification to obtain amplified products;

(3) performing genetic typing detection and sequencing on the amplified products to obtain a type of the molecular marker of poultry to be detected;

(4) judging poultry laying traits based on the type of the molecular marker.

In a further improvement, the amplified products have nucleotide sequences as shown in SEQ ID NO. 2.

In a further improvement, both the forward amplified product and the reverse amplified product of the site where the molecular marker is located have a length greater than 100 bp; a difference of the length between the forward amplified product and the reverse amplified product of the site where the molecular marker is located is greater than 20 bp.

In a further improvement, sequences of the specific amplimers are as follows:

Forward primer:
GGGACAGGGCCATACAGTTT;
Reverse primer:
TCAGTACTCCCCTGCTCATACA.

In a further improvement, the method for detecting the type of the molecular marker is as follows: digesting the PCR amplified products by an Nde I enzyme to obtain enzyme-digested products, and detecting the enzyme-digested products by electrophoresis, where if the enzyme-digested products contain:

(1) a band, the type of the molecular marker is GG;

(2) two bands, the type of the molecular marker is TT; and

(3) three bands, the type of the molecular marker is TG.

In a further improvement, the step of judging poultry laying traits based on the type of the molecular marker includes the specific steps of:

(1) if the type of the molecular marker of poultry to be detected is GG, the poultry laying traits are optimal;

(2) if the type of the molecular marker of poultry to be detected is TT, the poultry laying traits are poor;

(3) if the type of the molecular marker of poultry to be detected is TG, the poultry laying traits are ordinary.

The present disclosure further provides a method for screening poultry with excellent laying traits by the above molecular marker, including steps of extracting total DNA of poultry tissue cells, performing typing detection on genotypes of the poultry using the molecular marker, and choosing an individual poultry with a genotype of GG, namely, which is the poultry variety with excellent laying traits.

The present disclosure has the following beneficial effects: a molecular marker for identifying poultry laying traits based on an OVR gene, an identification method and an application thereof are provided. The present disclosure can make up the shortcomings of the conventional detection method, and has less experimental steps, short period, common reagents used and lower costs. The type of the molecular marker is identified in the poultry genome to judge the egg laying capacity of an individual poultry via molecular screening at the early stage, which provides direct technical means for the breeding of poultry egg laying capacity. By early breeding, the present disclosure reduces the feeding costs, and improve the egg laying level of an individual poultry at a genetic level, and quicken the development of genetic breeding.

### BRIEF DESCRIPTION OF THE DRAWINGS

FIG. 1 is a diagram showing agarose gel electrophoresis of PCR amplified products of partial samples;

FIG. 2 is a diagram showing digestion agarose gel electrophoresis of the PCR amplified products of partial samples;

FIG. 3 shows a polymorphic site determined by DNA sequences of pooling samples.

### DESCRIPTION OF THE EMBODIMENTS

The present application will be further described in detail with reference to the accompanying drawings below. It is necessary to point that the following detailed embodiments are merely used to further describe the present application, but not construed as limiting the protection scope of the present application. A person skilled in the art may make some nonessential improvements and adjustments on the present disclosure based on the disclosure of the above application.

1. Materials

Methods applied in the example are conventional methods known by a person skilled in the art, and reagents and other materials used are commercially available products, unless otherwise specified.

2. Methods

2.1 Obtaining the Polymorphic Site of Poultry OVR Gene

2.1.1 Extraction and Detection of Genomic DNA

250 female Wanxi white goose in total were selected as test materials and subjected to venous blood collection from the wings; a blood DNA extraction kit produced by Sangon Biotech was used to extract the total DNA in the venous blood samples of poultry wings. Specific steps are referring to the operating manual of the kit.

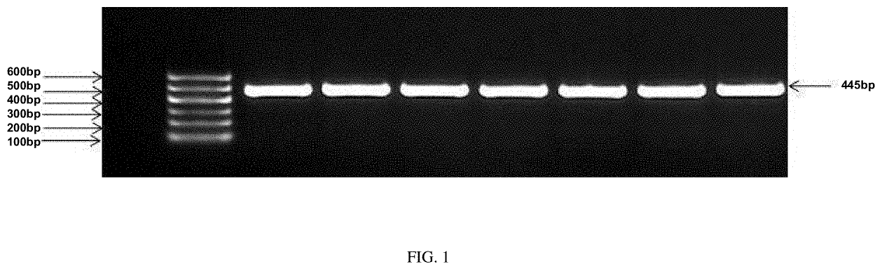

NanoDrop2000 was used to measure the DNA concentration and OD value. DNA was detected by 1.5% agarose gel electrophoresis. The results are shown in FIG. 1. The extracted genomic DNA has better quality, single and clear main band.

2.1.2 Primer Design

The complete genomic sequences of the goose oocyte were downloaded on the National Center of Biotechnology Information (NCBI, http://www.ncbi.nlm.nih.gov/) with accession number of MK446725.1 to find out the OVR gene and upstream sequences thereof from the genome database, as shown in SEQ ID NO.1. Partial DNA sequences of the OVR gene as shown in SEQ ID NO.1 served as a template. During the process of primer design, the SNP site should be disposed in the middle position as much as possible, thus avoiding the occurrence of a hairpin structure, a primer dimer, mispairing and the like and achieving an optimal primer sequences. The primer sequences are shown below:

F1:
(SEQ ID NO. 3)
GGGACAGGGCCATACAGTTT
R1:
(SEQ ID NO. 4)
TCAGTACTCCCCTGCTCATACA

The PCR product amplified by the primers had a length of 445 bp, including the molecular marker site of the G/T mutation at the site −399bp upstream of the transcription initiation site of the OVR gene.

2.1.3 PCR Amplification

The target fragment of the OVR gene was subjected to PCR amplification reaction by the synthesized specific sequencing primers; the PCR amplification system was as follows:

| Component | Amount | |
|---|---|---|
| DNA template | 1 | μL |
| Forward primer | 0.1 | μL |
| Reveres primer | 0.1 | μL |
| Mix | 7 | μL |
| ddH₂O | 7 | μL |
| Total | 15.2 | μL |

PCR amplification conditions: the target fragment was pre-denatured for 5 min at 95° C. , denatured for 30 s at 95° C. , re-denatured for 30 s at 58-62° C. , and elongated for 45 s at 72° C. with 30-35 cycles, and finally, elongated for 5 min at 72° C.

2.1.4 Detection on the PCR Amplified Products

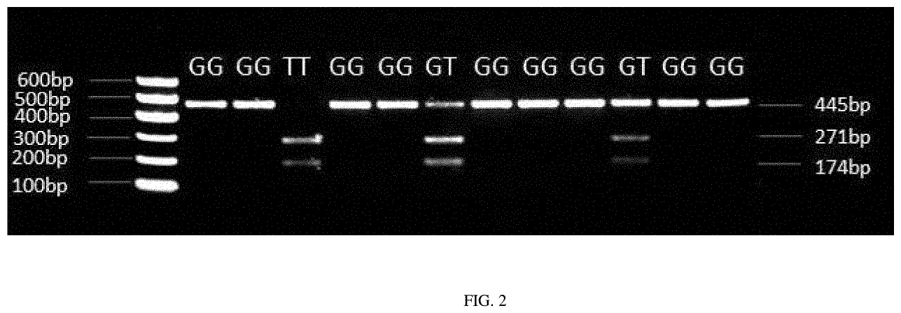

An agarose gel having a concentration of 1% was prepared for electrophoresis detection for 40 min at 100 V to obtain a band having a length of about 445 bp which was consistent to the predicted length. The results of the PCR amplified products are shown in FIG. 2. A DNA recovery kit from Dalian TaKaRa was applied to recover the PCR amplified products. The obtained PCR amplified products were sent to Shanghai Sangon Biotech for sequencing. The sequences are shown in SEQ ID NO.2, and are consistent to the predicted results.

2.1.5 Genotype Detection

The PCR amplified products were digested by an Nde I enzyme to obtain enzyme-digested products, and the enzyme-digested products were detected by electrophoresis. The results are shown in SEQ ID NO.2. Different genotypes were screened according to the results of the images photographed:

(1) if a band was contained, the type of the genotype was GG;

(2) if two bands were contained, the type of the genotype was TT; and

(3) if three bands were contained, the type of the genotype was TG.

2.1.6 DNA verification sequencing

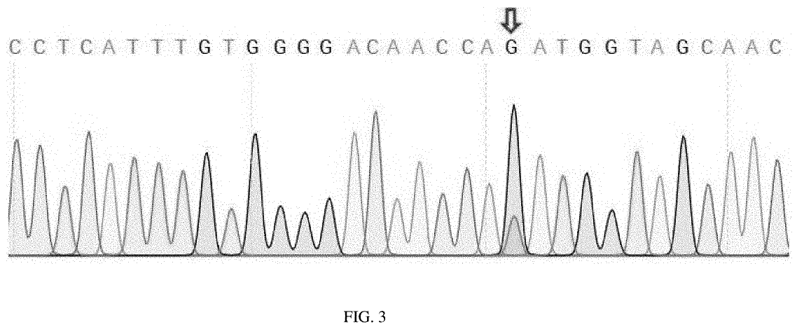

An individual was respectively subjected to sequencing alignment directed to the three genotypes. The sequencing alignment diagram is shown in FIG. 3; in the sequencing result, G mutates into T, and the arrow indicates the position of mutation, which is consistent to the predicted result.

2.2 Analysis on the Molecular Marker and on the Correlation of the Molecular Marker to the Laying Traits

2.2.1 Gene and Genotype Frequency

To determine the correlation of the molecular marker of the present disclosure to the important phenotypic characters of poultry, 250 Wanxi white goose in 2.1.1 and 250 Yangzhou goose picked were used as test materials.

2.2.2 Statistic Analysis

SAS(9.2) software was used to analyze the correlation of the genetic loci to laying traits. The data was firstly subjected to descriptive statistical analysis to make statistics on the egg production of the total 250 Wanxi white female goose and 250 Yangzhou female goose during two consecutive egg laying periods. Results are respectively shown in Tables 1-2.

TABLE 1
Records on the egg laying of each
genotype of Wanxi white goose OVR

| Genotype | GG | GT | TT |
|---|---|---|---|
| Frequency (%) | 91.74 | 7.39 | 0.87 |
| Egg production in the | 28 | 19 | 11 |
| first year (pcs.) | | | |
| Egg production in the | 36 | 23 | 13 |
| second year (pcs.) | | | |

TABLE 2
Records on the egg laying of each
genotype of Yangzhou goose OVR

| Genotype | GG | GT | TT |
|---|---|---|---|
| Frequency (%) | 94.06 | 5.31 | 0.63 |
| In-season (October to | 52 | 28 | 16 |
| April of the following | | | |
| year) | | | |
| Out-of-season (April to | 43 | 22 | 14 |
| October) | | | |

As can be seen from Tables 1-2, the results show that the egg production of the genotype GG is obviously higher than that of the GT and TT; the laying traits of the type TT are the worst, indicating that the molecular marker of the present disclosure is correlated to the poultry laying traits, and it is feasible to detect the poultry laying traits by means of the molecular marker.

The above examples merely indicate several embodiments of the present disclosure, and are described more specifically, but are not thus construed as limiting the protection scope of the present disclosure. It should be indicated that a person skilled in the art can further make several deformations and improvements within the inventive concept. Moreover, these deformations and improvements shall fall within the protection scope of the present disclosure.

1. A molecular marker for identifying poultry laying traits based on an OVR gene, wherein the molecular marker is G or T; the molecular marker is located at −399bp upstream of a transcription initiation site of an oocyte vitellogenesis receptor (OVR) gene of poultry.

2. The molecular marker for identifying poultry laying traits based on an OVR gene according to claim 1, wherein the molecular marker is located at a 417th site of a nucleotide sequence as shown in SEQ ID NO.1.

3. An application of the molecular marker for identifying poultry laying traits based on an OVR gene of claim 1, in identifying poultry laying traits.

4. A method for identifying poultry laying traits by using the molecular marker for identifying poultry laying traits based on an OVR gene of claim 1, comprising the following steps:
(1) extracting poultry blood and total DNA of any one tissue;
(2) serving OVR nucleotide sequences upstream and downstream of the site where the molecular marker is located as templates to design specific amplimers; serving the total DNA as a template and using the specific amplimers for PCR amplification to obtain amplified products;
(3) detecting a type of the molecular marker on the amplified products; and
(4) judging poultry laying traits based on the type of the molecular marker.

5. The method for identifying poultry laying traits by using the molecular marker for identifying poultry laying traits based on an OVR gene according to claim 4, wherein both the forward amplified product and the reverse amplified product of the site where the molecular marker is located have a length greater than 100 bp; a difference of the length between the forward amplified product and the reverse amplified product of the site where the molecular marker is located is greater than 20 bp.

6. The method for identifying poultry laying traits by using the molecular marker for identifying poultry laying traits based on an OVR gene according to claim 5, where sequences of the specific amplimers are as follows:
Forward primer:
GGGACAGGGCCATACAGTTT;
Reverse primer:
TCAGTACTCCCCTGCTCATACA.

7. The method for identifying poultry laying traits by using the molecular marker for identifying poultry laying traits based on an OVR gene according to claim 6, wherein the specific amplimers have nucleotide sequences as shown in SEQ ID NO.2.

8. The method for identifying poultry laying traits by using the molecular marker for identifying poultry laying traits based on an OVR gene according to claim 4, wherein the method for detecting the type of the molecular marker is as follows: digesting the PCR amplified products by an Nde I enzyme to obtain enzyme-digested products, and detecting the enzyme-digested products by electrophoresis, wherein if the enzyme-digested products contain:
(1) a band, the type of the molecular marker is GG;
(2) two bands, the type of the molecular marker is TT; and
(3) three bands, the type of the molecular marker is TG.

9. The method for identifying poultry laying traits by using the molecular marker for identifying poultry laying traits based on an OVR gene according to claim 8, wherein the step of judging poultry laying traits based on the type of the molecular marker includes the following specific steps:
(1) if the type of the molecular marker of poultry to be detected is GG, the poultry laying traits are optimal;
(2) if the type of the molecular marker of poultry to be detected is TT, the poultry laying traits are poor;
(3) if the type of the molecular marker of poultry to be detected is TG, the poultry laying traits are ordinary.

10. A method for screening poultry with excellent laying traits by the molecular marker of claim 1, comprising steps of extracting total DNA of poultry tissue cells, performing typing detection on a genotype of the poultry by using the molecular marker, and choosing an individual poultry with a genotype of GG, namely, which is the poultry variety with excellent laying traits.

11. The application of claim 3, wherein the molecular marker is located at a 417th site of a nucleotide sequence as shown in SEQ ID NO.1.

12. The method of claim 4, wherein the molecular marker is located at a 417th site of a nucleotide sequence as shown in SEQ ID NO.1.

13. The method of claim 10, wherein the molecular marker is located at a 417th site of a nucleotide sequence as shown in SEQ ID NO.1.
